(19) United States
(12) Patent Application Publication
(10) Pub. No.: US 2015/0031732 A1
(43) Pub. Date: Jan. 29, 2015

(54) FEBUXOSTAT SOLID DISPERSION

(71) Applicants: Poonam Kaushik, Ghaziabad (IN); Ram Thaimattam, Hyderabad (IN); Mohan Prasad, Gurgaon (IN)

(72) Inventors: Poonam Kaushik, Ghaziabad (IN); Ram Thaimattam, Hyderabad (IN); Mohan Prasad, Gurgaon (IN)

(73) Assignee: RANBAXY LABORATORIES LIMITED, New Delhi, Delhi (IN)

(21) Appl. No.: 14/111,332

(22) PCT Filed: Apr. 16, 2012
(86) PCT No.: PCT/IB12/51892

§ 371 Date: Sep. 17, 2014

(30) Foreign Application Priority Data
Apr. 15, 2011 (IN) 1120/DEL/2011

(51) Int. Cl. A61K 31/426; A61K 47/32; B29B 13/06; A61K 9/10

(52) U.S. Cl. CPC A61K 31/426; A61K 9/10; A61K 47/32; B29B 13/065
USPC 514/365; 264/12

(57) ABSTRACT

The present invention provides a solid dispersion of febuxostat, processes for its preparation, pharmaceutical compositions comprising it and its use for the chronic management of hyperuricemia in patients with gout.

### FIELD OF THE INVENTION

The present invention provides an amorphous solid dispersion of febuxostat, processes for its preparation, pharmaceutical compositions comprising it and its use for the chronic management of hyperuricemia in patients with gout.

### BACKGROUND OF THE INVENTION

Febuxostat is a non-purine xanthine oxidase inhibitor known from U.S. Pat. No. 5,614,520. It is chemically 2-[3-cyano-4-(2-methylpropoxy)phenyl]-4-methylthiazole-5-carboxylic acid having the structure as represented by Formula I.

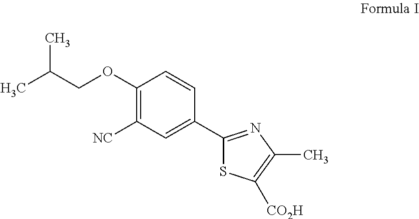 [SMILES: Cc1nc(-c2ccc(OCC(C)C)c(C#N)c2)sc1C(=O)O]

Febuxostat is marketed in the United States under the brand name Uloric® for the chronic management of hyperuricemia in patients with gout.

Amorphous form of febuxostat is disclosed in U.S. Pat. No. 6,225,474. Besides this, several other crystalline forms of febuxostat are known in literature. Solid dispersions of febuxostat are not disclosed in literature.

### SUMMARY OF THE INVENTION

The present invention provides an amorphous solid dispersion of febuxostat, processes for its preparation, pharmaceutical compositions comprising it and its use for the treatment of gout. The solid dispersion of the present invention improves the stability of the amorphous state of febuxostat.

A first aspect of the present invention provides an amorphous solid dispersion of febuxostat and a carrier.

A second aspect of the present invention provides a process for preparing the amorphous solid dispersion of febuxostat and a carrier comprising dissolving febuxostat and carrier in a solvent and removing the solvent from the solution.

A third aspect of the present invention provides a pharmaceutical composition comprising an amorphous solid dispersion of febuxostat and one or more pharmaceutically acceptable carriers, diluents or excipients.

A fourth aspect of the present invention provides use of an amorphous solid dispersion of febuxostat for chronic management of hyperuricemia in patients with gout.

### BRIEF DESCRIPTION OF THE FIGURES

FIG. 1: X-ray diffraction pattern of amorphous solid dispersion of febuxostat with polyvinyl pyrrolidone.

FIG. 2: X-ray diffraction pattern of amorphous solid dispersion of febuxostat with polyvinyl pyrrolidone on keeping at stability at ambient conditions for about 1 month.

### DETAILED DESCRIPTION OF THE INVENTION

Various embodiments and variants of the present invention are described hereinafter.

The term “solid dispersion”, as used herein, refers to systems having small solid-state particles of one phase dispersed in another solid-state phase. More particularly, amorphous solid dispersion of the present invention comprises febuxostat dispersed in a carrier in solid state. Amorphous solid dispersion of the present invention may be prepared by melting or solvent methods or by a combination of melting and solvent methods.

The term “ambient temperature”, as used herein, refers to temperature in the range of about 20° C. to about 35° C.

Febuxostat to be used for the preparation of the amorphous solid dispersion of the present invention may be obtained by any of the methods known in the literature such as those described in U.S. Pat. Nos. 5,614,520; 7,541,475; and U.S. Publication No. 2009/0203919, which are incorporated herein by reference. Febuxostat, to be used as starting material for the preparation of the amorphous solid dispersion of the present invention, may be obtained as a solution directly from a reaction in which it is formed and used as such without isolation or it may be isolated from the reaction mixture in which it is formed and then used for the preparation of the amorphous solid dispersion.

Examples of carriers to be used for the preparation of the amorphous solid dispersion of the present invention may include polyvinyl pyrrolidone (PVP), polyethylene glycol (PEG), polyvinyl alcohol (PVA), crospovidone, starch, pectin, pullulan, mannan, gelatin, gum arabic, a dextrin, a cyclodextrin, agar, a polyoxysorbitan fatty acid ester, an alginate or cellulose derivatives. Examples of cellulose derivatives may include hypromellose (HPMC), hydroxypropyl cellulose (HPC), hypromellose phthalate (HPMCP), hydroxypropyl methylcellulose acetate, hydroxypropyl methylcellulose acetate succinate cellulose (HPMCAS), ethyl cellulose, hydroxyethyl cellulose, methyl cellulose, carmellose (CMC), carmellose sodium (CMC-Na), carmellose calcium (CMC-Ca), croscarmellose sodium and low-substituted hydroxypropyl cellulose (L-HPC).

The amorphous solid dispersion of the present invention may be prepared by reacting about 0.5 to about 5 equivalents of polyvinyl pyrrolidone per equivalent febuxostat in solvent at ambient temperature to reflux temperature of solvent. Isolation of the solid dispersion may be carried out by quickly removing the solvent from the solution and drying. Removal of the solvent may be carried out by distillation at a temperature of about 50° C. to 80° C., by spray drying or agitated thin film drying. Drying may be carried out using any suitable method such as drying under reduced pressure, vacuum tray drying, air drying or a combination thereof at about 40° C. to 70° C. for about 4 hours to 8 hours.

Solvent(s) to be used for the preparation of the amorphous solid dispersion of the present invention may be selected from the group comprising of alcohols, carboxylic acids, chlorinated hydrocarbons, ketones, amides, sulphoxides, ethers, water or mixtures thereof. Examples of alcohols may include methanol, ethanol, 1-propanol, 1-butanol or 2-butanol. Examples of carboxylic acids may include formic acid, acetic acid or propionic acid. Examples of chlorinated hydrocarbons may include dichloromethane or chloroform. Examples of ketones may include acetone, dimethyl ketone, ethyl methyl ketone or methyl iso-butyl ketone. Examples of ethers may include diethyl ether, ethyl methyl ether, di-isopropyl ether, tetrahydrofuran or 1,4-dioxane. Examples of amides may include N,N-dimethylformamide or N,N-dimethylacetamide. Examples of sulphoxides may include dimethyl sulfoxide or diethyl sulphoxide. Examples of cyclic ethers may include tetrahydrofuran.

In one embodiment of the present invention, the amorphous solid dispersion may be prepared by dissolving febuxostat and polyvinyl pyrrolidone in an alcohol, removing the alcohol and drying. In another embodiment of the present invention, the amorphous solid dispersion may be prepared by dissolving febuxostat and polyvinyl pyrrolidone in methanol, removing methanol and drying. In a preferred embodiment of the present invention, the amorphous solid dispersion may be prepared by dissolving febuxostat and polyvinyl pyrrolidone in methanol, distilling the solvent from the solution using a Buchi rotavapor set at a temperature of about 65° C. and about 250 revolutions per minute (rpm) under reduced pressure, drying for about 10 minutes followed by vacuum tray drying at about 55° C. for about 6 hours. In another preferred embodiment, the amorphous solid dispersion may be prepared by dissolving febuxostat and polyvinyl pyrrolidone in methanol followed by spray drying using a spray dryer supplied with nitrogen gas at a feed pump rate of about 6 mL/minute. The inlet temperature of the spray dryer may be maintained at about 80° C. to 140° C. and outlet temperature may be maintained at about 35° C. to 65° C.

The amorphous solid dispersion of the present invention is retained for a long period of time under ambient conditions and is physically stable.

The amorphous solid dispersion of febuxostat and a carrier of the present invention may be administered as part of a pharmaceutical composition for the chronic management of hyperuricemia in patients with gout. Accordingly, in a further aspect, there is provided a pharmaceutical composition comprising an amorphous solid dispersion of febuxostat and a carrier and one or more diluents(s) or excipient(s). Amorphous solid dispersion of febuxostat and a carrier of the present invention may conventionally be formulated into tablets, capsules, suspensions, dispersions, injectables and other pharmaceutical forms. Any suitable route of administration may be employed, for example, peroral or parental.

In the foregoing section, embodiments are described by way of examples to illustrate the processes of invention. However, these are not intended in any way to limit the scope of the present invention. Several variants of the examples would be evident to persons ordinarily skilled in the art which are within the scope of the present invention.

### Method

X-ray diffraction pattern was recorded using an Panalytical Expert PRO with Xcelerator as the detector, 0.02 as step size and 3-40° 20 as range.

Spray drying was carried out using a Buchi Mini Spray Drier B-290; air inlet temperature was maintained at about 80° C. to about 140° C. and the outlet temperature was maintained at about 35° C. to about 65° C.

### EXAMPLES

Preparation of solid dispersion of febuxostat with polyvinyl pyrrolidone.

### Example 1

Febuxostat (5.01 g) and polyvinyl pyrrolidone (5.26 g) were dissolved in methanol (250 mL) by heating at about 65° C. The clear solution was fed into a spray dryer at a feed pump rate of about 6 mL/minute. The inlet temperature was maintained at about 120° C. and the outlet temperature was maintained at about 45° C. Solid material was dried in a vacuum tray dryer at about 50° C. for about 4 hours to obtain a solid dispersion of febuxostat with polyvinyl pyrrolidone.

Yield: 4.89 g

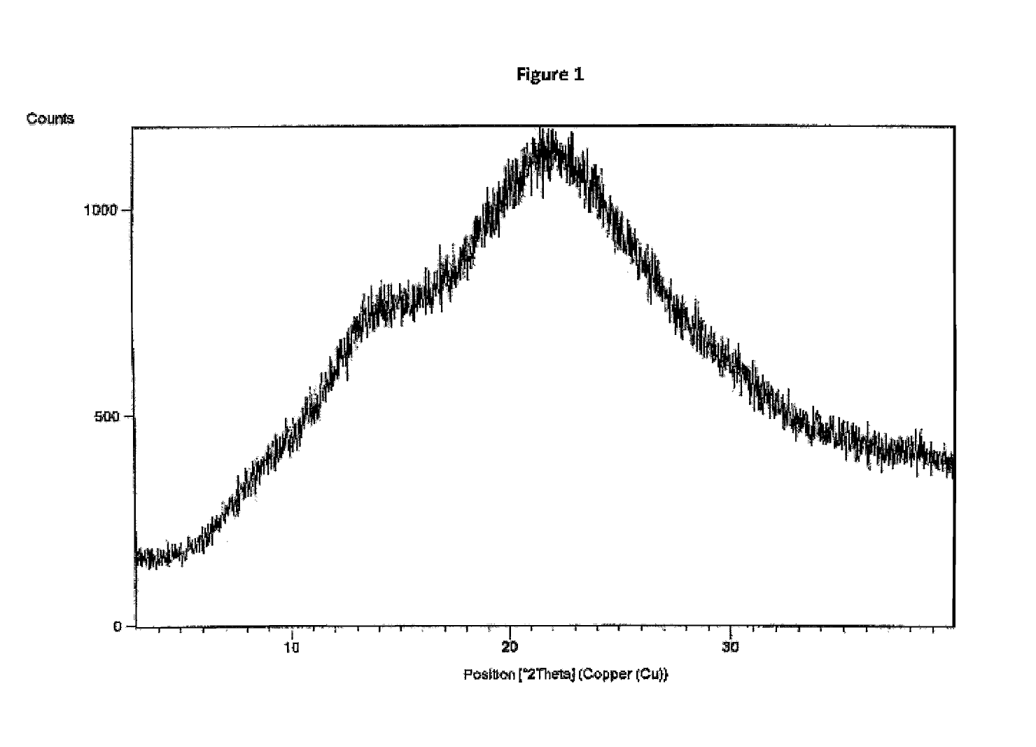

FIG. 1 depicts the X-ray diffraction pattern of the solid dispersion of febuxostat with polyvinyl pyrrolidone.

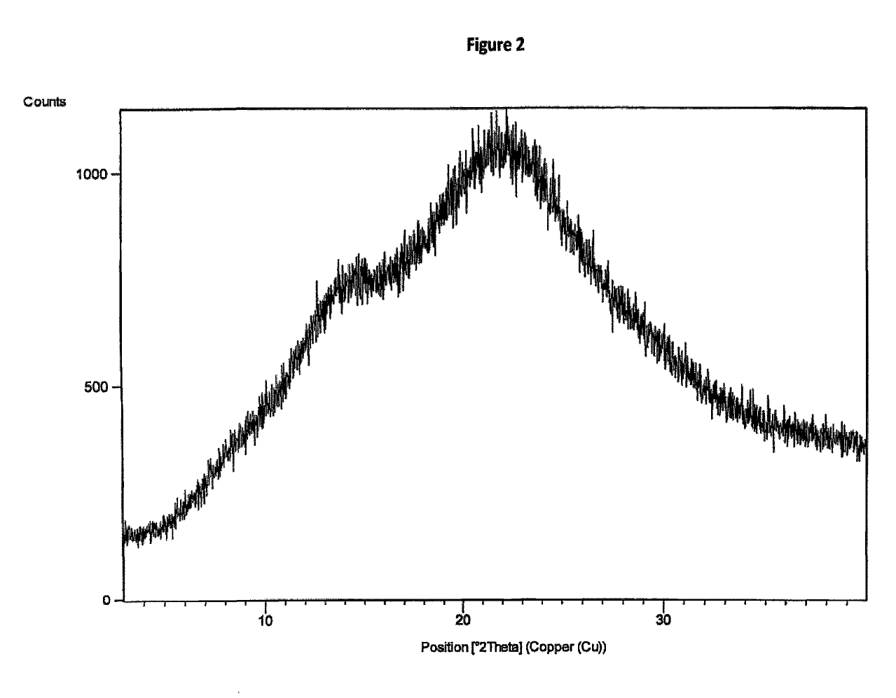

FIG. 2 depicts the X-ray diffraction pattern of the solid dispersion of febuxostat with polyvinyl pyrrolidone stored at ambient conditions for about 1 month.

### Example 2

Febuxostat (1.2 g) and polyvinyl pyrrolidone (1.1 g) were dissolved in methanol (60 mL). A clear solution was obtained. Solvent was distilled off using a Buchi rotavapor set at about 65° C. and about 250 rpm under reduced pressure. Solid material was dried under these conditions for about 10 minutes followed by drying in a vacuum tray dryer at about 55° C. for about 6 hours to obtain a solid dispersion of febuxostat with polyvinyl pyrrolidone.

Yield: 1.47 g

### Example 3

Febuxostat (0.99 g) and polyvinyl pyrrolidone (0.99 g) were dissolved in methanol (60 mL). A clear solution was obtained. Solvent was distilled off using a Buchi rotavapor set at about 65° C. and about 250 rpm under reduced pressure. Solid material was dried under these conditions for about 10 minutes followed by drying in a vacuum tray dryer at about 55° C. for about 6 hours to obtain a solid dispersion of febuxostat with polyvinyl pyrrolidone.

Yield: 1.20 g

We claim:

1. An amorphous solid dispersion of febuxostat and a carrier.

2. A process for preparing an amorphous solid dispersion of febuxostat and a carrier comprising dissolving febuxostat and carrier in solvent and removing the solvent from the solution.

3. The solid dispersion according to claim 1 or claim 2, wherein the carrier is selected from polyvinyl pyrrolidone (PVP), polyethylene glycol (PEG), polyvinyl alcohol (PVA), crospovidone, starch, pectin, pullulan, mannan, gelatin, gum arabic, a dextrin, a cyclodextrin, agar, a polyoxysorbitan fatty acid ester, an alginate or cellulose derivatives.

4. The process according to claim 2, wherein the solvent is selected from alcohols, carboxylic acids, chlorinated hydrocarbons, ketones, amides, sulphoxides, ethers, water or mixtures thereof.

5. The process according to claim 2, wherein dissolution of febuxostat and carrier in solvent is carried out at ambient temperature to reflux temperature of the solvent.

6. The process according to claim 2, wherein removal of solvent is carried out by spray drying.

7. The process according to claim 2, wherein removal of solvent is carried out by distillation at about 50° C. to 80° C.

8. Pharmaceutical composition comprising amorphous solid dispersion of febuxostat and one or more pharmaceutically acceptable carrier(s), diluent(s) or excipient(s).

9. Use of amorphous solid dispersion of febuxostat for chronic management of hyperuricemia in patients with gout.
